SEQUENCE LISTING (18 sequences)

SEQ ID NO: 1 - DNA, 666 bp, Artifical sequence
  cDNA sequence of Mad1 isoform 1
atggcggcggcggttcggatgaacatccagatgctgctggaggcggccgactatctggagcggcgggagagagaagctgaacatggttatgcctccatgttaccatacaataacaaggacagagatgccttaaaacggaggaacaaatccaaaaagaataacagcagtagcagatcaactcacaatgaaatggagaagaatagacgggctcatcttcgcttgtgcctggagaagttgaaggggctggtgccactgggacccgaatcaagtcgacacactacgttgagtttattaacaaaagccaaattgcacataaagaaacttgaagattgtgacagaaaagccgttcaccaaatcgaccagcttcagcgagagcagcgacacctgaagaggcagctggagaagctgggcattgagaggatccggatggacagcatcggctccaccgtctcctcggagcgctccgactccgacagggaagaaatcgacgttgacgtggagagcacggactatctcacaggtgatctggactggagcagcagcagtgtgagcgactctgacgagcggggcagcatgcagagcctcggcagtgatgagggctattccagcaccagcatcaagagaataaagctgcaggacagtcacaaggcgtgtcttggtctctaa

SEQ ID NO: 2 - protein, 221 aa, Artificial Sequence
  Mad1 isoform 1 amino acid sequence
MAAAVRMNIQMLLEAADYLERREREAEHGYASMLPYNNKDRDALKRRNKSKKNNSSSRSTHNEMEKNRRAHLRLCLEKLKGLVPLGPESSRHTTLSLLTKAKLHIKKLEDCDRKAVHQIDQLQREQRHLKRQLEKLGIERIRMDSIGSTVSSERSDSDREEIDVDVESTDYLTGDLDWSSSSVSDSDERGSMQSLGSDEGYSSTSIKRIKLQDSHKACLGL

SEQ ID NO: 3 - DNA, 636 bp, Artificial Sequence
  cDNA sequence of Mad1 isoform 2
atggcggcggcggttcggatgaacatccagatgctgctggaggcggccgactatctggagcggcgggagagagaagctgaacatggttatgcctccatgttaccatacaataacaaggacagagatgccttaaaacggaggaacaaatccaaaaagaataacagcagtagcagacgggctcatcttcgcttgtgcctggagaagttgaaggggctggtgccacttggacccgaatcaagtcgacacactacgttgagtttattaacaaaagccaaattgcacataaagaaacttgaagattgtgacagaaaagccgttcaccaaatcgaccagcttcagcgagagcagcgacacctgaagaggcagctggagaagctgggcattgagaggatccggatggacagcatcggctccaccgtctcctcggagcgctccgactccgacagggaagaaatcgacgttgacgtggagagcacggactatctcacaggtgatctggactggagcagcagcagtgtgagcgactctgacgagcggggcagcatgcagagcctcggcagtgatgagggctattccagcaccagcatcaagagaataaagctgcaggacagtcacaaggcgtgtcttggtctctaa

SEQ ID NO: 4 - protein, 211 aa, Artificial Sequence
  Mad1 isoform 2 amino acid sequence
MAAAVRMNIQMLLEAADYLERREREAEHGYASMLPYNNKDRDALKRRNKSKKNNSSSRRAHLRLCLEKLKGLVPLGPESSRHTTLSLLTKAKLHIKKLEDCDRKAVHQIDQLQREQRHLKRQLEKLGIERIRMDSIGSTVSSERSDSDREEIDVDVESTDYLTGDLDWSSSSVSDSDERGSMQSLGSDEGYSSTSIKRIKLQDSHKACLGL

SEQ ID NO: 5 - protein, 86 aa, Artificial Sequence
  (DH-KR) Mad1*C75S-E77K-G81R-C111  S-D112H which are aa 56-141
MSRSTHNEMEKNRRAHLRLSLKKLKRLVPLGPESSRHTTLSLLTKAKLHIKKLEDSHRKAVHQIDQLQREQRHLKRQLEKLGGSGC

SEQ ID NO: 6 - protein, 86 aa, Artificial Sequence
  (DV-KR) Mad1*C75S-E77K-G81R-C111S-D112V which are aa 56-141
MSRSTHNEMEKNRRAHLRLSLKKLKRLVPLGPESSRHTTLSLLTKAKLHIKKLEDSVRKAVHQIDQLQREQRHLKRQLEKLGGSGC

SEQ ID NO: 7 - protein, 86 aa, Artificial Sequence
  (DN-KR) Mad1*C75S-E77K-G81R-C111S-D112N which are aa 56-141
MSRSTHNEMEKNRRAHLRLSLKKLKRLVPLGPESSRHTTLSLLTKAKLHIKKLEDSNRKAVHQVDQLQREQRHLKRQLEKLGGSGC

SEQ ID NO: 8 - protein, 86 aa, Artificial Sequence
  Mad1*DN-KR-K which are aa 56-141
MSRSTHNEMEKNRKAHLRLSLKKLKRLVPLGPESSRHTTLSLLTKAKLHIKKLEDSNRKAVHQVDQLQREQRHLKRQLEKLGGSGC

SEQ ID NO: 9 - protein, 86 aa, Artificial Sequence
  Mad1*DN-KR-H which are aa 56-141
MSRSTHNEMEKNRHAHLRLSLKKLKRLVPLGPESSRHTTLSLLTKAKLHIKKLEDSNRKAVHQVDQLQREQRHLKRQLEKLGGSGC

SEQ ID NO: 10 - protein, 86 aa, Artificial Sequence
  Mad1*DN-KR-TYR  which are aa 56-141 and aa 61 can also be Val
MSRSTHNEMEKNRRAHLRLSLKKLKRLVPLGPESSRHTTLSLLTKAKLHIKKLEDSNRKAIYQVDQLQREQRHLKRQLEKLGGSGC

SEQ ID NO: 11 - protein, 86 aa, Artificial Sequence
  Mad1*DN-R-KR  which are aa 56-141 and aa 61 can also be Val
MSRSTHNEMERNRRAHLRLSLKKLKRLVPLGPESSRHTTLSLLTKAKLHIKKLEDSNRKAIHQVDQLQREQRHLKRQLEKLGGSGC

SEQ ID NO: 12 - protein, 86 aa, Artificial Sequence
  Mad1*DN-R-KR-TYR  which are aa 56-141 and aa 61 can also be Val
MSRSTHNEMERNRRAHLRLSLKKLKRLVPLGPESSRHTTLSLLTKAKLHIKKLEDSNRKAIYQVDQLQREQRHLKRQLEKLGGSGC

SEQ ID NO: 13 - protein, 86 aa, Artificial Sequence
  Mad1* polypeptide which are aa 56-141 and aa 61 can also be Val
MSRSTHNEMEKNRRAHLRLSLKKLKRLVPLGPESSRHTTLSLLTKAKLHIKKLEDSNRKAIHQVDQLQREQRHLKRQLEKLGGSGC

SEQ ID NO: 14 - protein, 86 aa, Artificial Sequence
  Mad1* polypeptide which are aa 56-141 and aa 61 can also be Val
MSRSTHNEMEKNRRAHLRLSLKKLKRLVPLGPESSRHTTLSLLTKAKLHIKKLEDSNRKAIHQIDQLQREQRHLKRQLEKLGGSGC

SEQ ID NO: 15 - DNA, 18 bp, Artificial Sequence
  DNA probe  fluorescently labeled
gagtagcacgtgctactc

SEQ ID NO: 16 - DNA, 18 bp, Artificial Sequence
  DNA probe  fluorescently labeled
gagtagggatccctactc

SEQ ID NO: 17 - DNA, 37 bp, Artificial Sequence
  PCR probe
cttgtccctgaagaagttgaagcgcctggtgccactg

SEQ ID NO: 18 - DNA, 37 bp, Artificial Sequence
  PCR probe
cagtggcaccaggcgcttcaacttcttcagggacaag

(19) United States
(12) Patent Application Publication
(10) Pub. No.: US 2020/0299341 A1
(43) Pub. Date: Sep. 24, 2020

(54) POLYPEPTIDES AND USES THEREOF FOR THE TREATMENT OF CANCER

(71) Applicant: SOCIETE DE COMMERCIALISATION DES PRODUITS DE LA RECHERCHE APPLIQUEE SOCPRA SANTE ET HUMAINES, S.E.C., Sherbrooke (CA)

(72) Inventors: Pierre LAVIGNE, Sherbrooke (CA); Martin MONTAGNE, Sherbrooke (CA); Loika MALTAIS, Sherbrooke (CA)

(73) Assignee: SOCIETE DE COMMERCIALISATION DES PRODUITS DE LA RECHERCHE APPLIQUEE SOCPRA SANTE ET HUMAINES, S.E.C., Sherbrooke (CA)

(21) Appl. No.: 16/086,179

(22) PCT Filed: Mar. 24, 2017
(86) PCT No.: PCT/CA2017/050375

§ 371 Date: Sep. 18, 2018

Related U.S. Application Data
(60) Provisional application No. 62/312,737, filed on Mar. 24, 2016.

(51) Int. Cl. C07K 14/47

(52) U.S. Cl. CPC C07K 14/47; A61K 38/00; C07K 2319/09

(57) ABSTRACT

It is provided isolated polypeptides comprising the b-HLH-LZ domain of Mad 1, wherein the polypeptides comprise mutation of the amino acid residue D1 12 of isoform 1, and wherein said polypeptide penetrates into cells useful for treating cancer such as lung cancer.

### CROSS-REFERENCE TO RELATED APPLICATIONS

This application includes a sequence listing in electronic format which is being concurrently filed herewith. This application also claims priority from U.S. provisional application Ser. No. 62/312,737 filed on Mar. 24, 2016. The content of the sequence listing and of the priority application is herewith incorporated in its entirety.

### TECHNICAL FIELD

It is provided an isolated polypeptide comprising the b-HLH-LZ domain of Mad1 that penetrates into cells and is useful for treating cancer.

### BACKGROUND

Cancer refers to more than one hundred clinically distinct forms of the disease. Almost every tissue of the body can give rise to cancer and some can even yield several types of cancer. The twelve major cancers are prostate, breast, lung, colorectal, bladder, non-Hodgkin's lymphoma, uterine, melanoma, kidney, leukemia, ovarian, and pancreatic cancers.

Lung cancer has now become the 5^{th} leading cause of death worldwide. The World Health Organization (WHO) distinguishes two types of lung cancer: small cell (SCLC) and non-small cell lung cancer (NSCLC). Both lead to the development of deadly adenocarcinomas. Two of the genes most frequently mutated in NSCLC are those coding for the Epidermal Growth Factor Receptor (EGFR), a receptor tyrosine kinase, and KRAS, a small GTPase. 10-15% of patients with advanced lung adenocarcinomas bear an activating mutation in the EGFR gene, while another 30% have an activating mutation in the KRAS gene. Although patients with mutations in the EGFR gene initially respond well to tyrosine kinase inhibitors (TKI) such as gefinitib or erlotinib, tumor cells often develop resistance to those inhibitors after 10-16 months. However, patients harboring mutations in KRAS are refractory to TKI. In fact, to date, there is no specific treatment available for these patients. Hence, there is an imperative need for new treatments of NSCLC that do not give rise to resistance and that are effective against KRAS-driven adenocarcinomas. Although mutations in EGFR and KRAS are mutually exclusive, both lead to the sustained activity of the PI3K-AKT and/or RAS-MEK-ERK pathways. These pathways contribute to the phosphorylation, stabilization and sustained activation of c-Myc, a basic Helix-Loop-Helix-Leucine Zipper (b-HLH-LZ) transcription factor, which is the gene product of the proto-oncogene c-myc. In addition, c-myc is mutated, amplified and translocated in many other cancers. However, c-Myc protein does not need to be mutated to be oncogenic as its expression is often deregulated as a consequence of activating mutations in other oncogenes.

Recent studies have demonstrated that the genome-wide binding of c-Myc in cancer cells correlates more with the presence of RNA polymerase II (Pol II) than with the presence of E-box sequences. On the one hand, physiological levels of c-Myc will lead to its preferential location at E-box sequences found at target genes promoters and enhancers that carry its normal functions in cell cycle entry and metabolism. Binding of c-Myc to E-Box sequences at promoters and enhancers results in the acetylation of Histones (e.g. H3K27Ac), opening of chromatin and the phosphorylation of the c-Terminal tail of RNA Pol II. On the other hand, at supra-physiological or oncogenic levels, c-Myc spills over all active promoters and enhancers (where Pol II is located), and amplifies transcription irrespective of the presence of an E-Box. Amplification of gene networks promoting proliferation and the eviction of apoptosis leads to the addiction of tumor cells to supra-physiological and oncogenic levels of c-Myc. This phenomenon referred to as “addiction to the oncogene” represents a clear opportunity to explore in order to develop new therapeutic strategies with minimal side-effects and a wide application in oncology.

It has been suggested that systemic inhibition of c-Myc, even transient, could lead to the selective apoptosis of tumor cells without causing serious and/or irreversible effects in healthy tissues. It was demonstrated that the inhibition of c-Myc by systemic expression of a transgene coding for a designed b-HLH-LZ c-Myc dominant negative (Omomyc) leads to the complete clearance of tumors in a mouse model of KRAS^{G12D}-driven NSCLC, with only minor and tolerable side-effect in normal tissues (e.g. reversible attrition of the intestinal epithelium). Strikingly, systemic Myc inhibition by Omomyc does not lead to tumor cell resistance even after prolonged treatment. The use of Omomyc proteins for the treatment of cancers is also described in international PCT patent publication WO 2014/180889.

There is thus a need for new therapies, methods and compositions for the treatment of cancers including, but not limited to, lung cancer.

### BRIEF SUMMARY

It is provided an isolated polypeptide comprising the b-HLH-LZ domain of Mad1, wherein the polypeptide penetrates into cells.

In an embodiment, the polypeptide is (i) an amino acid sequence corresponding to a fragment of SEQ ID NO: 2 or SEQ ID NO: 4; or (ii) an amino acid sequence corresponding to a mutated SEQ ID NO: 2 or SEQ ID NO: 4.

In another embodiment, the polypeptide described herein comprises amino acid 58-67 of SEQ ID NO: 2.

In a further embodiment, the polypeptide described herein comprises a mutation at position 112 of SEQ ID NO: 2.

In an embodiment, the mutation is selected from the group consisting of D112H, D112N, and D112V.

In a further embodiment, the polypeptide described herein comprises a mutation at position 102 of SEQ ID NO: 4.

In another embodiment, the mutation is selected from the group consisting of D102H, D102N, and D102V of SEQ ID NO: 4.

In an additional embodiment, the polypeptide described herein comprises a mutation for generating a Nuclear Localization Signal (NLS).

In an embodiment, the NLS comprises an amino acid sequence corresponding to K-(K/R)-X-(K/R).

In another embodiment, the NLS is generated by introducing mutations at positions 77 and 81 of SEQ ID NO: 2.

In an embodiment, the NLS comprises the amino acid sequence K77-K78-L79-K80-R81.

In another embodiment, the NLS is generated by introducing mutations at positions 67 and 71 of SEQ ID NO: 4.

In another embodiment, the NLS comprises the amino acid sequence K67-K68-L69-K70-R71.

In a further embodiment, the polypeptide described herein further comprises a mutation at position 75 of SEQ ID NO: 2, a mutation at position 111 of SEQ ID NO: 2, a mutation at position 65 of SEQ ID NO: 4, a mutation at position 101 of SEQ ID NO: 4, and/or a C-terminal tail.

In an embodiment, the polypeptide described herein comprises a mutation C75S (SEQ ID NO: 2), a mutation C65S (SEQ ID NO: 4), a mutation C111S (SEQ ID NO: 2) and/or a mutation C101S (SEQ ID NO: 4).

In another embodiment, the tail comprises the amino acid sequence GSGC.

In an additional embodiment, the polypeptide described herein comprises a mutation increasing resistance to proteases degradation.

In an embodiment, the polypeptide described herein comprises a mutation at a consensus furin PCSK7 or subtilisin-like cleavage site.

In a further embodiment, the polypeptide described herein comprises a mutation at position 69 of SEQ ID NO: 2.

In another embodiment, the polypeptide described herein comprises a mutation R69K (SEQ ID NO: 2) or a mutation R69H (SEQ ID NO: 2).

In an additional embodiment, the polypeptide described herein further comprises a mutation at position 117 of SEQ ID NO: 2, a mutation at position 119 of SEQ ID NO: 2, a mutation at position 107 of SEQ ID NO: 4, or a mutation at position 109 of SEQ ID NO: 4.

In another embodiment, the polypeptide described herein comprises a mutation H117Y (SEQ ID NO: 2), a mutation 1119V (SEQ ID NO: 2), H107Y (SEQ ID NO: 4), or a mutation 1109V (SEQ ID NO: 4).

It is also provided an isolate polypeptide comprising an amino acid sequence selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14.

It is also provided a pharmaceutical composition comprising a polypeptide as defined herein, and a pharmaceutically acceptable vehicle.

It is further provided an isolated or purified nucleic acid molecule encoding a polypeptide as defined herein.

It is additionally provided a genetically modified cell expressing a polypeptide as defined herein.

In an additional embodiment, the polypeptide described herein is for the treatment of a cancer.

It is provided the use of a polypeptide as defined herein for the treatment of a cancer.

It is provided the use of a polypeptide as defined herein in the manufacture of a medicament for the treatment of a cancer.

It is also provided a method for treating of cancer in a subject in need thereof, the method comprising administering to the subject a polypeptide as defined herein.

In an embodiment, the cancer is Human Lung cancer, small-cells lung cancer, Human Colon cancer, Human Glioblastoma, Human Lymphoma, Human Dermal skin fibroblast cancer, Human Myeloid fibroblast, or Breast Cancer.

In an embodiment, the polypeptide as defined herein further comprises chemically linked a second molecule.

In another embodiment, the second molecule is at least one of a fluorophore, a therapeutic molecule, a polypeptide and a nucleic acid.

In an additional embodiment, the therapeutic molecule is an anticancer agent.

In an embodiment, the anticancer agent is auristatin E, paclitaxel, docetaxel, topotecan, decarbazine, doxorubicin, daunorubicin, cyclophosphamide, busulfex, busulfan, vinblastine, vincristine, bleomycin, etoposide, topotecan, irinotecan, taxotere, taxol, 5-fluorouracil, methotrexate, gemcitabine, cisplatin, carboplatin or chlorambucil.

### BRIEF DESCRIPTION OF THE DRAWINGS

Reference will now be made to the accompanying drawings.

FIG. 1 illustrates Mad1 homodimeric b-HLH-LZs endocytosis in HeLa cells showing confocal photomicrographs of HeLa cells incubated for 1 hour in cell culture medium containing 10 μM of either (A) Mad1*DN-TexasRed, (B) Mad1*DV-TexasRed or (C) Max*, wherein white arrows point to nuclei stained with DAPI.

FIG. 2 illustrates the impact of E77K and G81R mutations on Mad1* endocytosis and cellular localization in H23 and WI-38 cells, wherein WI-38 and H23 cells were incubated for 2 hours in respective cell culture medium containing 10 μM of (A) Mad1*DN, (B) Mad1*DV, (C) Mad1*DN-KR or (D) Mad1*DV-KR, wherein all proteins were labelled with TexasRed fluorophore on their c-terminal cysteine end, and white arrows point to nuclei stained with DAPI (small images).

FIG. 3 illustrates metabolic assays evaluating the relative cell survival following treatments with Max*, Mad1*DN and Mad1*DV. WST-1 assays performed 24 to 72 hours following a single treatment of (A) HeLa cells (B) H23 cells and (C) WI-38 cells with 50 M (HeLa cells) or 25 μM (H23 and WI-38 cells) of Max* (black), Mad1*DN (grey) and Mad1*DV (white), wherein the percentage of cell survival was calculated relatively to control cells treated with PBS 1×, error bars indicate the standard deviation (N=3).

FIG. 4 illustrates the impact of E77K and G81R mutations on the potency and specificity of Mad1* treatments, wherein WST-1 assays performed 48 hours following a single treatment of (A) H23 cells (5 μM) or (B) WI-38 cells (25 μM) with Mad1* initial versions DN and DV (black) or Mad1*DN-KR and DV-KR (grey), wherein the percentage of cell survival was calculated relatively to control cells treated with PBS 1×; cell survival percentages are superimposed for easier comparison; and error bars indicate the standard deviation (N=3), **** P<0.0001.

FIG. 5 illustrates dose-response inhibition of WI-38 and H23 cells by Mad1*-KR mutants. WST-1 assays performed 48 hours following single treatments ranging from 100 nM to 100 μM of Mad1*DN-KR (squares), DV-KR (triangles) and DH-KR (circles), wherein curves for WI-38 (full lines) and H23 (dotted lines) cells were obtained by non-linear regression (sigmoidal dose-response), error bars represent standard deviation, N=3, and IC₅₀ values are listed in Table 1.

FIG. 6 illustrates representation of the therapeutic window offered by Mad1*DN-KR. WST 1 assays performed 48 hours following single treatments of Mad1*DN-KR ranging from 100 nM to 100 μM, wherein curves for WI-38 (full lines) and H23 (dotted lines) cells were obtained by non-linear regression (sigmoidal dose-response), the box representing the therapeutic window of Mad1*DN-KR, and error bars represent standard deviation, N=3.

FIG. 7 illustrates caspase-3 activation in lung cancer cells (H23), showing confocal photomicrographs of WI-38 and H23 cells treated for 18 hours with (A) PBS 1× (control) and (B) Mad1*DN-KR, wherein white arrow point to cleaved Caspase-3 detected by anti-cleaved Caspase-3 antibody, and nuclei were stained with DAPI (small images).

FIG. 8 illustrates comparative thermal denaturations of Mad1*DN and its KR analog (Mad1*DN-KR) recorded by circular dichroism at 222 nm.

FIG. 9 illustrates the comparative heterodimerization of Max (Max*WT) with Mad1*DN or Mad1*DN-KR by thermal denaturations curves recorded by circular dichroism at 222 nm showing in (A) the denaturation of Max*WT 16 μM+Mad1*DN 16 μM; in (B) Max*WT 16 μM+Mad1*DN-KR 16 μM; and in (C) heterodimers with Max*WT of Mad1*DN or Mad1*DN-KR.

FIG. 10 illustrates comparison of homodimeric and heterodimeric binding of specific (Ebox) and non-specific DNA determined by fluorescence anisotropy showing in (A) homodimeric DNA binding and in (B) heterodimeric DNA binding.

FIG. 11 illustrates the ex vivo stability assay of Cy5 labeled Mad1*DN-KR in commercial mouse plasma, wherein Mad1*DN-KR, labeled by NHS-Cy5 on lysine residues, was mixed to commercial mouse plasma at 37° C. for up to 24 hours, fractions were separated by SDS-PAGE and visualized by fluorescence imaging in (A) or by Western Blot in (B) using polyclonal Mad1* antibodies, the semi-quantification of bands in (A) is represented in (C).

FIG. 12 illustrates the ex vivo stability assay of Texas-Red labeled Mad1*DN-KR in commercial mouse plasma, wherein Mad1*DN-KR, labeled by maleimide-Texas-Red on the last and only cysteine residue, was mixed to commercial mouse plasma at 37° C. for up to 24 hours, fractions were separated by SDS-PAGE and visualized by fluorescence imaging in (A) or by Western Blot in (B) using polyclonal Mad1* antibodies.

FIG. 13 illustrates metabolic assays evaluating the relative cell survival following treatments with Max*, Omomyc and Mad1*DN-KR. WST-1 assays performed 48 hours following a single treatment of A549 cells with 25 μM of polypeptides, wherein the percentage of cell survival was calculated relatively to control cells treated with PBS 1×, error bars indicate the standard deviation (N=3).

FIG. 14 illustrates the dose-response inhibition of WI-38 and U87 cells by Mad1*DN-KR mutants, wherein WST-1 assays performed 48 hours following single (full lines) or repeated (dotted lines) treatments ranging from 100 nM to 100 μM of Mad1*DN-KR, curves for WI-38 (circles) and U87 (triangles) cells were obtained by non-linear regression (sigmoidal dose-response), and error bars represent standard deviation, N=3.

FIG. 15 illustrates the evaluation of the blood residence half-life following the injection of a dose IV (caudal vein) of Mad1*DN-KR (24.3 mg/kg) in a healthy BALB/c mouse, wherein in (A) 3.5 μl plasma samples (2, 5, 10, 20, 40 and 60 minutes; wells 3 to 8 respectively)) were loaded on a 15% SDS/PAGE which include a sample before injection (TO) loaded on well 9, and Western blot was done using anti-Mad1 IgY (chicken; Immune BioSolution) and HRP-anti-chicken (alpaca; Immune BioSolution) and revealed by ECL after an exposure of 5 minutes on a photographic film, quantification of the bands was done using Image J and plotted on the graph presented in (B) to estimate the blood residence half-life of Mad1*DN-KR using a non-linear fit (GraphPad Prism 7.0), error bars represent the standard deviation (SD) of the mean quantity calculated from the two positive controls (Mad1*DN-KR) of known quantity in the well 1 (0.035 μg) and well 2 (0.0065 μg).

### DETAILED DESCRIPTION

It is provided an isolated polypeptide comprising the b-HLH-LZ domain of Mad1, wherein the polypeptide penetrates into cells.

c-Myc and Max are members of a large network of b-HLH-LZ transcription factors that also includes L-Myc, N-Myc and proteins from the Mad family (Mad1, Mxi1, Mad3 and Mad4). The Myc and Mad proteins exert most of their transcriptional activities as Myc/Max and Mad/Max heterodimers. The Mad and Myc proteins do not readily homodimerize. The HLH-LZ domains are responsible for the specific heterodimerization of the Myc and Mad proteins with Max as well as for the reversible homodimerization of Max, while the basic region is responsible for DNA recognition through specific interactions in the DNA major groove. All the dimers of this network bind specific DNA sequences called the E-Box (CANNTG), with a preference for the canonical CACGTG sequence located near the core promoters of c-Myc target genes.

Deregulated and sustained c-Myc activity inhibits the transcription of cell cycle inhibitors and activates the transcription of pro-proliferative genes. Therefore, it is encompassed herein treatment methods comprising inhibiting or neutralizing c-Myc activity and/or c-Myc expression using the polypeptides and/or nucleic acid molecules as described herein.

It is thus described novel Mad1 polypeptides and these Mad1 polypeptides antagonize Myc-activated transcription by competing for Max and the E-box sequences. As such, the polypeptides encompassed herein are useful for the treatment of various cancers involving Myc, including but not limited to, lung cancer.

Mad1 is a protein of about 25 kDa. In humans, there are two isoforms of the protein having 221 amino acids (isoform 1) or 211 amino acids (isoform 2). The human cDNA sequence of Mad1 (isoform 1) is represented as SEQ ID NO: 1 and its accession number is Q05195 is cited under NCBI Gene ID 4084, NM_002357. The amino acid sequence of the Mad1 protein (isoform 1) is represented as SEQ ID NO: 2 and its accession number is Q05195. The second transcript isoform uses an alternate in-frame splice site in the 3′ coding region, compared to isoform 1. The resulting protein is 10 amino acids shorter than isoform 1 because it lacks amino acids 58 to 67 (corresponding to the basic domain residues) of SEQ ID NO: 2. The human cDNA sequence of Mad1 (isoform 2) is represented as SEQ ID NO: 3 and its accession number is Q05195-2 is cited under NCBI Gene ID 4084, NM_001202513.1. The amino acid sequence of the Mad1 protein (isoform 2) is represented as SEQ ID NO: 4 and its accession number is Q05195-2.

It is provided the successful demonstration that Mad1 polypeptide-based inhibitors selectively induce apoptosis in NSCLC and inhibit their proliferation much more potently than Max* or Omomyc without affecting normal lung fibroblasts across a wide range of concentrations.

As demonstrated recently, the b-HLH-LZ domain of Max (Max*) can spontaneously penetrate into HeLa cells and accumulate to their nuclei in order to inhibit c-Myc transcriptional activities (Montagne et al., 2012, PLoS One, 7(2): e32172). Mad1 is a transcription factor that dimerizes with Max via its b-HLH-LZ domain and inhibits c-Myc target genes expression by recruiting HDAC to their promoters and reverting euchromatin into hetero- and transcriptionally inactive chromatin. The polypeptides described in the previous publication were not contacted with living cells, let alone cancer cells, and it was unknown prior to the present disclosure that Mad1 polypeptides as encompassed herein can penetrate spontaneously into cells.

It was investigated whether Mad1 b-HLH-LZ domain (Mad1*) can spontaneously penetrate into cells. Since wild type Mad1 b-HLH-LZ domain is unable to homodimerize and bind DNA as a homodimer, two mutant versions of the b-HLH-LZ were used, Mad1*D112N and Mad1*D112V. These Mad1* mutants have the ability to homodimerize, heterodimerize and bind E-Box sequences in both forms. Texas Red™ fluorophores were covalently added to the C-terminal end of the proteins in order to visualize their endocytosis by immunofluorescence and confocal microscopy techniques. As shown, Mad1*D112N and Mad1*D112V indeed penetrate into HeLa cells and localize into cytoplasmic inclusions (most likely endosomes) one hour after incubation with 10 μM of proteins (FIGS. 1A and 1B).

Unlike Max*, Mad1 polypeptides do not seem to escape endosomes and consequently do not localize into the nucleus of HeLa cells (FIG. 1C). Therefore, E77K and G81R mutations in Helix I of Mad1*DN and DV were intended to increase the global charge of the proteins in order to facilitate their endosomal escape. Moreover, the E77K mutation allowed the creation of a nuclear localization signal (NLS) based on the monopartite consensus sequence K-(K/R)-X-(K/R). An additional mutation at position D112 for a Histidine was designed to maximize the endosomal escape since the monomeric form obtained by D112H mutation may potentiate the eviction from acidified endosomes. Monomeric, amphipathic and positively charged polypeptides like the present designed Mad1 polypeptides are most effective in causing the leakage of endosomes. However, this mutation still allows for homodimerization at cellular pH and preserves the interaction with Max protein. These new Mad1* polypeptide variants are named Mad1*DH/N/V-KR.

To better assess the characteristics of the present inhibitors on cancer cells, experiments were pursued on a cellular model of lung cancer. H23 cells from human Non-Small Cell Lung Cancer (NSCLC) were chosen for their mutation in the oncogene KRAS (G12D). This constitutively active mutation contributes to the activation and stabilization of c-Myc. Normal lung fibroblasts, WI-38, were used as control cells. Surprisingly, the initial versions of Mad1*, Mad1*DN and Mad1*DV, were already localizing to the nucleus of H23 cells (Non-Small Cell Lung Cancer) as well as WI-38 cells (normal lung fibroblasts) (FIGS. 2A and B). Although the E77K and G81R mutations did not seem to modify the endocytosis and localization kinetics of Mad1*DV-KR in the H23 and WI-38 cells, differences were observed for Mad1*DN-KR (FIGS. 2C and D). Indeed, the endocytosis of Mad1*DN-KR in WI-38 cells is slowed as the protein staining is localized to the cell membrane compared to a diffuse pattern in the cytoplasm and the nucleus of WI-38 cells incubated with Mad1*DN. No significant difference was observed in the H23 cells between Mad1*DN and DN-KR endocytosis.

Previously, it has been demonstrated that Max* can inhibit c-Myc transcriptional activities resulting in cell cycle arrest and apoptosis of HeLa cells (Montagne et al., 2012, PLoS One, 7(2): e32172). Herein it is described the testing to see whether Mad1* mutants would inhibit cell proliferation even more efficiently than Max* by inducing apoptosis and halting the cell cycle. This is exactly what is observed in a metabolic assay using WST-1 salt as a substrate. This assay allows the evaluation of the relative cell viability following treatments with Mad1* mutants compared to control cells treated only with PBS 1×.

FIG. 3A shows significantly less survival of HeLa cells following Mad1*DN treatment in comparison with Max* and almost no survival of HeLa cells treated with Mad1*DV. It is noted that the initial inhibition at 24 hours is sustained up to 72 hours following a single treatment at 50 μM. The same trend is observed in H23 lung cancer cells; albeit with a significant increase in potency (FIG. 3B). Treatment of normal lung fibroblasts (WI-38) does not lead to such an effect. Indeed WI-38 cells are much more viable when compared to lung cancer cells (H23). This demonstrates a specificity of action in cancer cells and confirms a therapeutic advantage of Mad1 polypeptides over Max* (FIG. 3C).

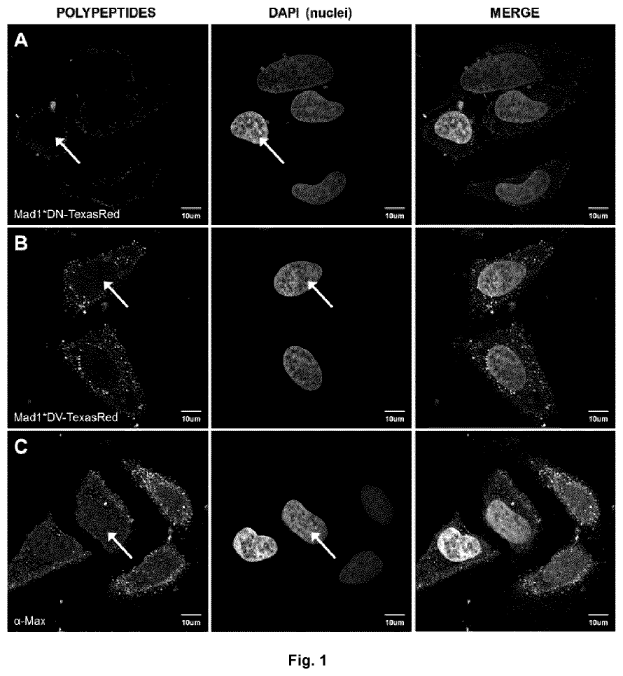

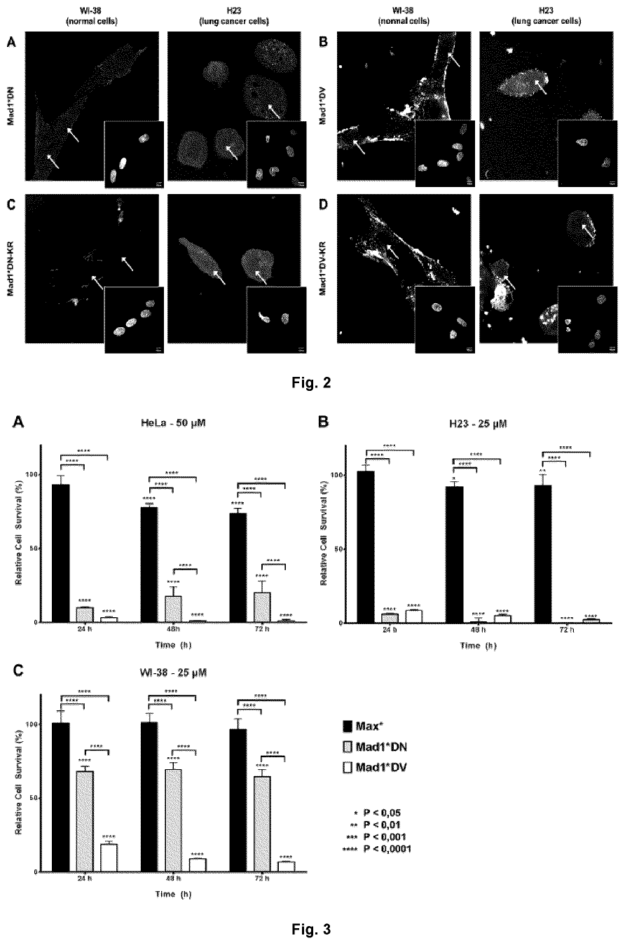

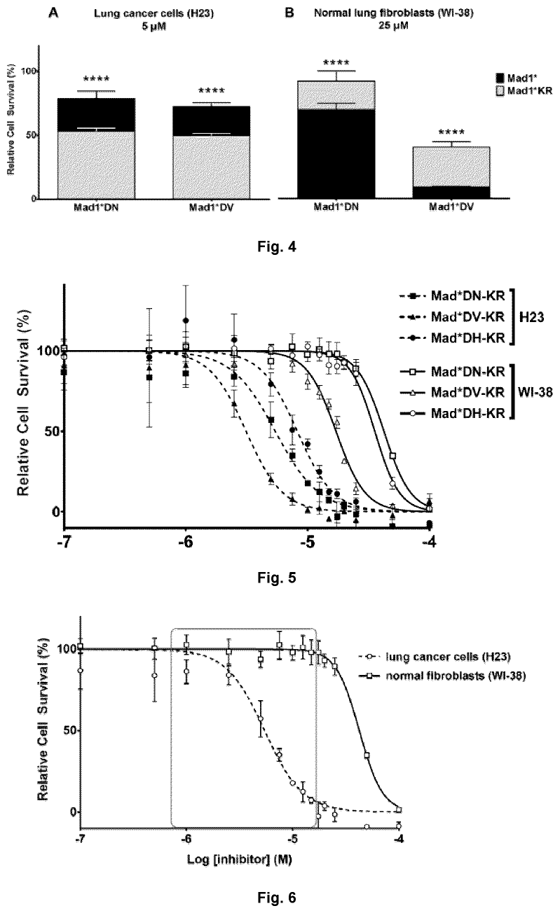

Accordingly, the new KR mutants (Mad1*DH/N/V-KR) were designed to promote a better endosomal escape and nuclear localization. Those mutations led to a selectivity of the Mad1* inhibitors towards lung cancer cells (H23 vs. WI-38). Indeed, FIG. 4A shows that survival of H23 cells following treatment with doses as low as 5 μM of Mad1*DN-KR or DV-KR is significantly decreased when compared to cells treated with Mad1*DN or DV. Thus, the KR mutants offer an improved potency over the Mad1* D112N or D112V initial versions for the inhibition of cell proliferation. On the other hand, an increase in normal fibroblasts survival was observed for both Mad1*DN-KR and DV-KR treatment, even at doses up to 25 μM (FIG. 4B). Taken together, these results indicate that the KR mutations improved the potency and the specificity of the present Mad1* inhibitors for cancer cells.

In order to define a potential therapeutic window for the present polypeptide inhibitors, the half maximal inhibitory concentrations (IC₅₀) was determined for KR mutants in lung cancer cells and normal fibroblasts. These values were calculated using the results of WST-1 assays performed 48 hours following single treatments with doses ranging from 0 to 100 μM (FIG. 5).

As depicted in Table 1, Mad1*DN-KR offers the largest therapeutic window with IC₅₀ of 5.44 μM and 42.43 μM in lung cancer and normal cells, respectively. It is also worth noting that H23 cell survival is almost totally inhibited at concentrations higher than 15 μM of Mad1*DN-KR, whereas the survival of WI-38 cells is not affected until doses reaching 20 μM (FIG. 6).

TABLE 1
IC50 values of the Mad1*KR mutants.

| | IC50 (μM) | | |
|---|---|---|---|
| Polypeptide | H23 | WI-38 | Ratio* |
| Mad1*DN-KR | 5.44 ± 1.55 | 42.43 ± 1.86 | 7.80 |
| Mad1*DV-KR | 3.13 ± 1.48 | 17.14 ± 2.07 | 5.47 |
| Mad1*DH-KR | 8.24 ± 1.66 | 35.70 ± 1.87 | 4.33 |

*Ratio of WI-38/H23 IC₅₀. Represent the therapeutic window width.

In order to further investigate the mechanism behind their impaired cell survival, an experiment was carried out to determine whether the cells undergo apoptosis upon treatment. For this purpose, phosphatidylserine in the external cell membrane was detected by flow cytometry, an indication of the induction of apoptosis. Using PE labelled Annexin-V along with 7-aminoactinomycine D (7-AAD, DNA marker) allows for the discrimination between early and late apoptosis.

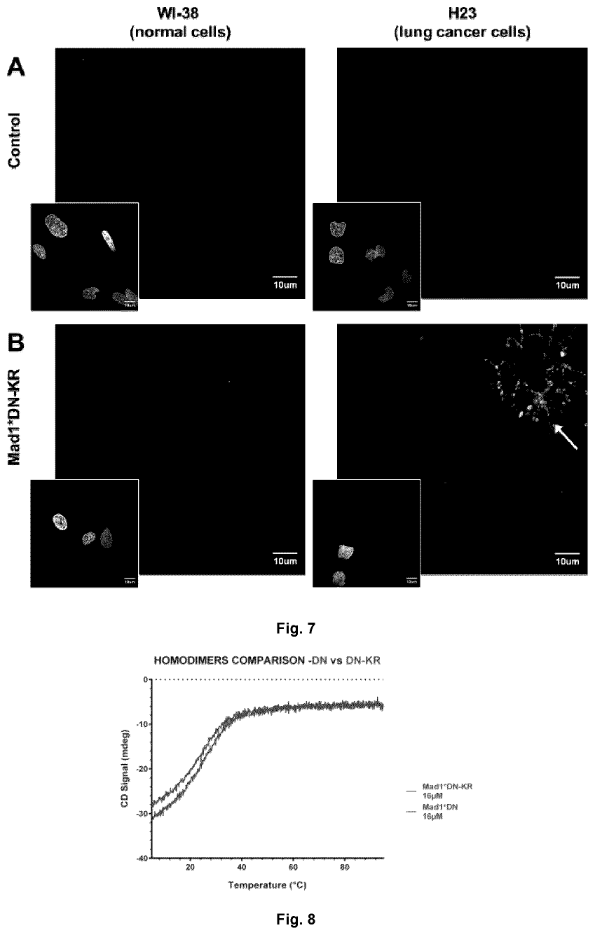

As shown in Table 2, the total percentage of cells undergoing apoptosis after treatment with Mad1*KR mutants differs slightly from cancer (H23) to normal (WI-38) cells. However, there is a significant difference between stages of apoptosis reached by the cells. Lung cancer cell populations in late apoptosis are much larger than those in normal fibroblasts. This suggests that the kinetic of apoptosis induction is faster in H23 cells. This hypothesis is confirmed by the immunodetection of cleaved Caspase-3, an executor Caspase, only in H23 cells treated with Mad1*KR mutants (FIG. 7).

TABLE 2
Apoptosis levels of cells treated with Mad1*KR mutants for 18 hours.

| | | | WI-38 | | | H23 | |
|---|---|---|---|---|---|---|---|
| | | | Apoptosis (%) | | | Apoptosis (%) | |
| Treatment | [ ] μM | Early | Late | Total | Early | Late | Total |
| PBS 1X | — | 5.4 | 2.4 | 7.8 | 4.5 | 5.0 | 9.5 |
| Staurosporine* | 2 | 39.0 | 7.5 | 46.5 | 24.4 | 16.6 | 41.0 |
| Mad1*DH-KR | | 19.2 | 2.4 | 21.6 | 13.3 | 12.1 | 25.4 |
| Mad1*DN-KR | 15 | 16.9 | 3.5 | 20.4 | 12.1 | 14.7 | 26.8 |
| Mad1*DV-KR | | 20.8 | 7.6 | 28.4 | 15.7 | 31.7 | 47.4 |

*Staurosporine is used as an apoptosis induction control.

Thus, it is demonstrated that b-HLH-LZs (including Mad1* mutants) are able to selectively inhibit proliferation and cause apoptosis in lung (NSCLC) cancer cells.

In its native form, Mad1 does not homodimerize significantly due to the presence of an aspartate at position 112 in its Leucine Zipper. The specific mutation of this amino acid by a valine, an asparagine or a histidine allows for the formation of homodimeric b-HLH-LZs with varying thermodynamic stability: D112V>D112N>D112H respectively. Mad1* mutants bind to E-box sequences in vitro with affinities correlating to the homodimers stabilities. These Mad1* mutants can also heterodimerize with Max, preventing its dimerization with c-Myc, and subsequent binding to E-box sequences in vitro.

When compared to the Max* proteins described previously (Montagne et al., 2012, PLoS One, 7(2): e32172), it was noticed that the Mad1* mutants are indeed more efficient than Max* at reducing the number of viable HeLa cells even though they did not seem to readily accumulate in the cell nuclei (FIGS. 1B, C and 3A). This indicates that the ability to both homodimerize and to retain the ability to heterodimerize with Max provides a clear advantage to Mad1*DH, DN and DV.

The tested Mad1* polypeptides are specific to cancer cells and the Mad1* mutants proved to be of much higher relevance than Max* (FIGS. 3B and C).

The improved Mad1*D112 mutants (called herein for example KR variants DN-KR, DH-KR and DV-KR) were engineered to favor endosomal escape and to promote nuclear localization in order to increase their potency. E77K and G81R mutations were designed to generate a nuclear localization signal (NLS), based on the monopartite consensus sequence (K-(K/R)-X-(K/R): E₇₇/K-K₇₈L₇₉-K₈₀-G₈₁/R), within the existent b-HLH-LZ domain without interfering with its activities. Moreover, mutations in this highly conserved Helix I of Mad1* might favor a better interaction with Max (i.e. sequestration). Indeed, adding basic residues in this hydrophobic and positively charged cluster of Mad1* may reduce Mad1* homodimeric HLH interaction hence facilitating the formation of Max/Mad* heterodimer.

Mad1*DN-KR, DH-KR and DV-KR penetrate and translocate to the nuclei of H23 cells without major difference compared to Mad1*DN and DV (FIG. 2). However, the specificity of inhibition was increased with the KR variants, the Mad1*DN-KR, DH-KR and DV-KR mutants showing a significant decrease for the potency of the polypeptide inhibitors in normal cells and a simultaneous increase in cancer cells (FIG. 4).

Furthermore, the IC₅₀ measured for Mad1*DN-KR (5.4 μM), DV-KR (3.1 μM) and DH-KR (8.2 μM) in H23 cells are significantly inferior to the one in WI-38 (42.3 μM, 17.1 μM and 35.7 μM respectively) (FIG. 5 and Table 1). This demonstrates a definite specificity for the reduction of the number of viable NSCLC cells, by a combination of cell cycle arrest and apoptosis. Although this experiment indicates that Mad1*DV-KR is more potent than DN-KR, the latter has a lower toxicity in normal cells and possesses a better therapeutic window (FIG. 6). The potentiating effect obtained from KR mutations could be attributable to the destabilization of the homodimeric Mad1 HLH, allowing for a better interaction for Max protein. Indeed, sustained HLH interaction with Max might contribute to the observed beneficial differences (Mad1*DN-KR>Mad1*DH-KR>Mad1*DV-KR).

The developed polypeptide inhibitors specifically induce apoptosis of H23 cancer cells without affecting WI-38 normal lung fibroblasts. This is corroborated with cleaved Caspase-3 staining, membrane blebbing, nuclear fragmentation and chromatin condensation of H23 cells (FIG. 7 and Table 2). These phenotypic apoptotic responses were undetectable on normal lung fibroblasts on a wide range of concentration, which correspond to a safe therapeutic window. Despite the fact that apoptosis rates determined by Annexin-V detection were equivalent in both cell lines, it is demonstrated that apoptosis in H23 cells is induced more rapidly considering the proportion of late apoptosis reached compared to WI-38 cells.

Accordingly, Mad1 mutants are able to selectively inhibit proliferation and cause apoptosis in lung (NSCLC) cancer cells. These results show that Mad1 mutants are very potent at eliminating cancer cells and more particularly at clearing lung tumors.

Proteins of the Mad family antagonize c-Myc-activated transcription by competing for Max and the E-box sequences. According to the present disclosure, the residue D112 located in the LZ of Mad1 can be exploited to induce homodimerization, while maintaining the ability to recognize Max. In addition to sequestering Max, Max/Mad endogenous full-length heterodimers recruit Histone DeACetylases (HDAC), through their mSin3 Interacting Domain (SID), to the c-Myc target gene promoters. This results in the deacetylation of histones, which in turn reverts transcriptionally active chromatin into more condensed and inactive chromatin. Whereas the transcription of c-Myc is activated by growth factors, the transcription of Mad proteins is activated by cytostastic factors (e.g. TGF-β). However, the activation of the PI3K-AKT and/or RAS-MEK-ERK pathways also lead to the phosphorylation and degradation of Mad proteins. Finally, the expression of Mad proteins is believed to participate to the terminal stages of differentiation.

It is also encompass Max, Mad2, Mad3, Mad4 and/or Mnt mutated polypeptides and homologs capable of penetrating spontaneously into cells, and the use thereof for the treatment of cancers. Useful polypeptides and homologs with anti-cancer activity can be obtained by those skilled in the art, using the present description, the information provided hereinafter in Tables 3-5 and routing testing and experimentation (e.g. for assaying or confirming desired biological activities).

TABLE 3
Alternative names and UniProt accession number of Max and Mad family members.

| | Max | Mad1 | Mad2 | Mad3 | Mad4 | Mnt |
|---|---|---|---|---|---|---|
| Accession number | P61244 | Q05195 | P50539 | Q9BW11 | Q14582 | Q99583 |
| | Protein Max | Protein MAD | Max-interacting | Max dimerization | Max dimerization | Max-binding protein |
| | | | protein 1 | protein 3 | protein 4 | MNT |
| | Myc Associated | Max dimerization | Max interactor 1 | Max dimerizer 3 | Max dimerizer 4 | Myc antagonist |
| | factor X | partner | | | | MNT |
| | Class D basic helix- | Max dimerizer 1 | Class C basic helix- | Class C basic helix- | Class C helix-loop- | Class D basic helix- |
| | loop-helix protein 4 | | loop-helix protein 11 | loop-helix protein 13 | helix protein 12 | loop-helix protein 3 |
| Protein Alternative | bHLHd4 | | bHLHc11 | bHLHc13 | bHLHc12 | bHLHd3 |
| names | | | | | | |
| | | | | Max-associated | Max-associated | Protein ROX |
| | | | | protein 3 | protein 4 | |
| | | | | Max-interacting | Max-interacting | |
| | | | | transcriptional | transcriptional | |
| | | | | repressor MAD3 | repressor MAD4 | |
| | | | | Myx | | |
| | MAX | MXD1 | MXI1 | MXD3 | MXD4 | MNT |
| Gene names | BHLHD4 | MAD | BHLHC11 | BHLHC13 | BHLHC12 | BHLHD3 |
| | | | | MAD3 | MAD4 | ROX |

TABLE 4
Comparative identity and similarity between Mad1 full length
protein and Max, Mad and Mnt proteins

| Full | | | | | | |
|---|---|---|---|---|---|---|
| Length | | Max | Mad2 | Mad3 | Mad4 | Mnt |
| Proteins | Parameters | (161aa) | (228aa) | (206aa) | (209aa) | (583aa) |
| Mad1 | Alignment | 221 | 237 | 230 | 226 | 583 |
| (221aa) | length | | | | | |
| | Identical | 36 | 127 | 64 | 78 | 41 |
| | residues | | | | | |
| | Similar residues | 30 | 34 | 19 | 25 | 30 |
| | Percent identity | 16.29% | 53.59% | 27.83% | 34.51% | 7.03% |
| | Percent | 29.86% | 67.93% | 36.09% | 45.58% | 12.18% |
| | similarity | | | | | |

TABLE 5
Comparative identity and similarity between Mad1 b-HLH-LZ
and Omomyc, Max, Mad and Mnt b-HLH-LZs

| b-HLH-LZ | | Max* | Omomyc* | Mad2* | Mad3* | Mad4* | Mnt* |
|---|---|---|---|---|---|---|---|
| Polypeptides | Parameters | (87aa) | (92aa) | (85aa) | (88aa) | (88aa) | (82aa) |
| Mad1* | Alignment length | 83 | 92 | 85 | 88 | 88 | 82 |
| (83aa) | Identical residues | 21 | 23 | 56 | 43 | 54 | 28 |
| | Similar residues | 16 | 20 | 12 | 8 | 10 | 11 |
| | Percent identity | 25.30% | 25.00% | 65.88% | 48.86% | 61.36% | 34.15% |
| | Percent similarity | 44.58% | 46.74% | 80.00% | 57.95% | 72.73% | 47.56% |

To measure the effect of the design on protein-protein interactions (homo-vs. heterodimerization) a circular dichroism (CD)-based approach was used (Montagne et al., 2005, Biochemistry, 44:12860-12869). According to this approach, the stability of a dimeric population is proportional to the melting temperature when heated.

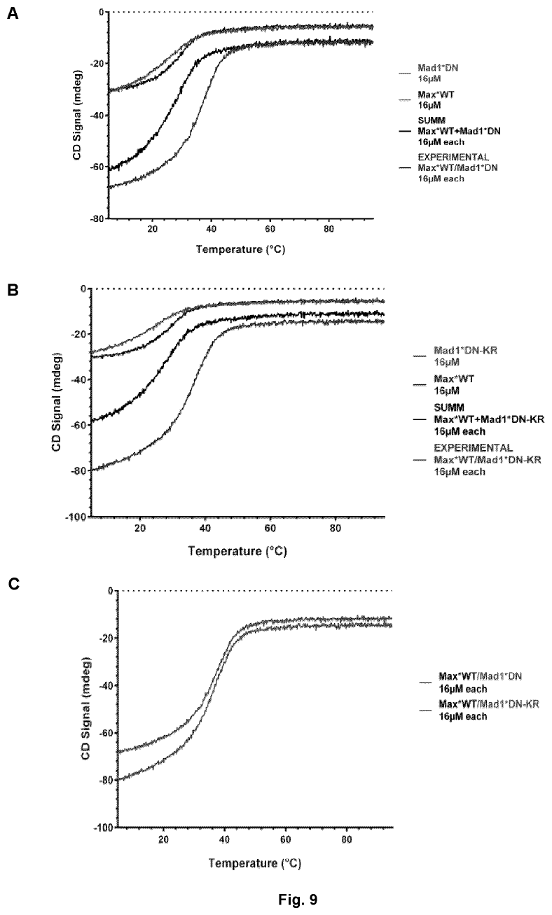

Central to the improvement of b-HLH-LZ inhibitors of c-Myc, such as Mad1*DN, is the optimization of their potential to sequester Max and bind specific (E-Box) and non-specific DNA as heterodimers (Mad1*DN/Max) and homodimers (Mad1*DN/Mad1*DN). With such a premise, the Mad1*DN-KR analog (SEQ ID NO: 7) was designed. The modifications were made with the objective to weaken the homodimerization by introducing electrostatic repulsions at the Mad1*DN-KR homodimer interface while introducing favorable electrostatic interactions at the interface of the Mad1*DN-KR/Max heterodimer. Moreover, because of the position of the R in the loop of Mad1*, it was anticipated that additional non-specific interaction with DNA would occur. In order to validate this hypothesis, the effect of the KR mutations on protein/protein and protein DNA interactions were quantified (FIGS. 8 and 9). FIG. 8 provides a comparative thermal denaturation of Mad1*DN and its KR analog (Mad1*DN-KR), as recorded by CD. As shown in FIG. 8, the melting temperature of Mad1*DN-KR denaturation is slightly lower than that of Mad1*DN. This indicates that the homodimer is weakened by the KR mutations in absence of DNA.

FIG. 9 provides thermal denaturation curves recorded by CD and compares heterodimerization of Max (Max*WT) with Mad1*DN or Mad1*DN-KR. As can been seen, the experimental curves of heterodimeric species show a good heterodimeric interaction of Max with either Mad1*DN (FIG. 9A) or Mad1*DN-KR (FIG. 9B). The melting temperature of Mad1*DN-KR/Max* denaturation is greater than that of Mad1*DN/Max* (FIG. 9C) indicating that the Mad1*DN-KR mutations allow for a more stable heterodimer with Max* in absence of DNA. In accordance, also with the premises of the designs of these polypeptides, it is seen on FIG. 9B that the melting temperature of Mad1*DN-KR/Max* heterodimer is increased compared to that of the Mad1*DN/Max* heterodimer.

To further validate the effect of our design on the DNA binding affinity (E-Box and non-specific DNA), a fluorescence anisotropy-based binding assay was used. In such a technique, double-stranded DNA probes are fluorescently labeled (E-Box: 5′-GAG/iFluorT/AGCACGTGCTACTC-3′ (SEQ ID NO: 15); non-specific: 5′-GAG/iFluorT/AGGGATCCCTACTC-3′ (SEQ ID NO: 16)) and titrated with increasing amount of protein(s). As the proteins (homo- or heterodimers) bind the fluorescent DNA probes, the anisotropy increases. The larger the anisotropy, the larger the amount of complex formed, which allows to determine the affinity (apparent biding constant) of the different dimers for the specific and non-specific DNA.

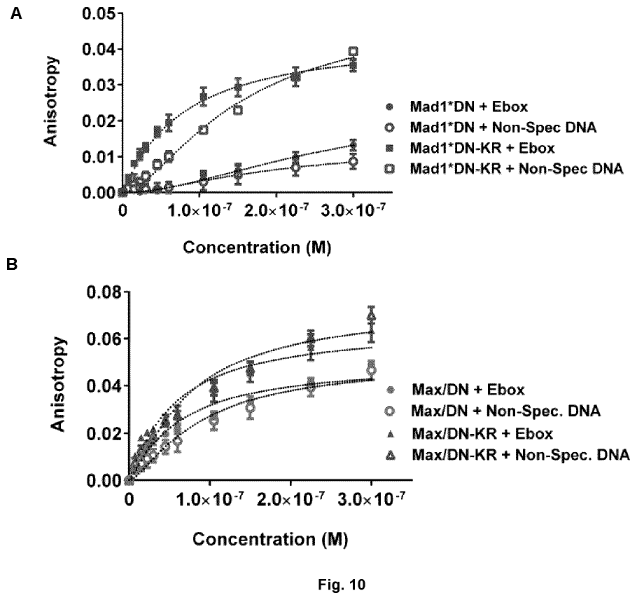

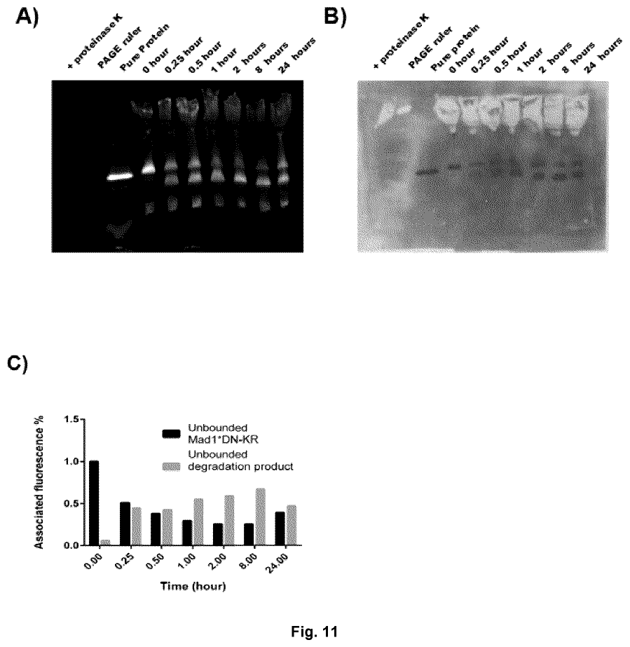

FIGS. 10A and 10B compares homodimeric and heterodimeric binding of specific (E-Box) and non-specific DNA. The apparent affinities of Mad1*DN-KR for both the E-box and non-specific probes are largely improved compared to the Mad1*DN counterparts. Mad1*DN-KR shows a better specific and non-specific homodimeric DNA binding when compared to Mad1*DN (FIG. 10A). DNA binding of Max/Mad1*DN-KR heterodimeric complex is improved compared to the Max/Mad1*DN heterodimeric complex (FIG. 10B).

Altogether, these results demonstrate an improved homodimeric DNA binding of Mad1*DN-KR over Mad1*DN and improved heterodimeric DNA binding of Max (Max*WT) with Mad1*DN-KR over Mad1*DN. Such improved binding supports the use of the polypeptides described herein in inhibition of c-Myc, particularly for the treatment of various cancers involving Myc.

Also central to the in vivo activity of Mad1* analogs as encompassed herein is their ability to resist to the degradation by proteases found in the serum. Resistance to plasma degradation was thus assessed as an initial estimation of the resistance of the Mad1* analogs described herein.

Briefly, 11 μL of Mad1*DN-KR (final concentration of 25 μM) was incubated in 0.1 mL of commercial mouse serum. The integrity of fluorescently labeled Mad1*DN-KR as a function of the incubation time was monitored by SDS-PAGE and visualized either by fluorescence (FIG. 11A) or by Western blot (FIG. 11B) using a specific antibody.

Ex vivo plasma stability results indicate that Mad1*DN-KR resists partially to plasma degradation and that it is stable for more than 24 hours in mouse plasma (see FIGS. 11A-C). Partial digestion occurs rapidly, but 40% of intact Mad1*DN-KR is still detected after a 24 hours incubation period at 37° C.

When considering SEQ ID NO: 7, a potential tetrabasic non-canonical cleavage site can be identified in the basic domain region of Mad1*DN-KR. This cleavage site corresponds to a known consensus furin, PCSK7 or subtilisin-like cleavage site (RX(R/K)R).

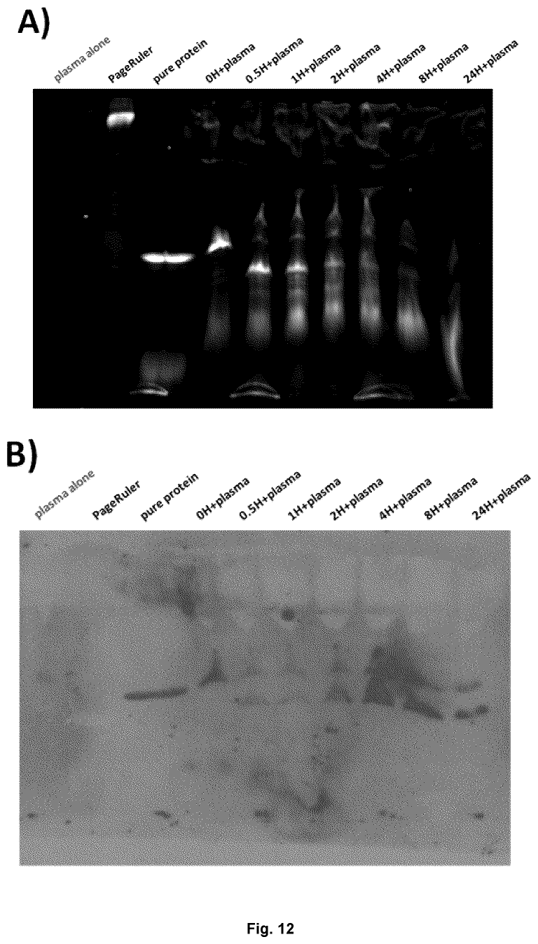

Studies were carried out to characterized the cleavage of Mad1*DN-KR. The protease(s) activity generates two fragments that are detectable by immunoblotting using Mad1* antibodies as shown on FIGS. 11B and 12B. One of the cleaved fragment (˜8.4 kDa) comprises amino acids 70-141, because it corresponds to the observed size of the cleaved fragment of Mad1*DN-KR labeled with Texas-Red on its only cysteine in C-terminal as shown on FIG. 11A. This ˜8.4 kDa fragment is still fluorescent, indicating that it corresponds to the C-terminal portion and that the cleavage occurs in the basic region of Mad1*DN-KR, most likely by furin/PCSK7/subtilisin-like serine proteases.

Therefore, it is predicted that resistance of the polypeptides as encompassed herein increases by modifying their amino acid sequence in order to remove cleavage site(s) that are recognized by proteases in plasma. In one embodiment, the cleavage site consists of the serine protease consensus motif (RX(R/K)R) that is present in the Mad1*DN-KR.

In one particular embodiment, the modification comprises a substitution of the last arginine (R) of the furin/PCSK7/subtilisin consensus recognition cleavage site, for instance by a lysine (K) or an histidine (H). Examples of such mutated polypeptides are: Mad1*DN-KR-K (SEQ ID NO: 8) and Mad1*DN-KR-H (SEQ ID NO: 9). These two mutations preserve (partially for Mad1*DN-KR-H) the global positive charge within the basic domain and contribute to preserve the cell penetration properties. They should also preserve the Max recognition/interaction and they should preserve the DNA binding ability of the homodimer (Mad1*DN-KR-K for example) and of the Max/Mad1*DN-KR-K heterodimers. The present invention encompasses any additional suitable mutation allowing to preserve the above-mentioned desired properties.

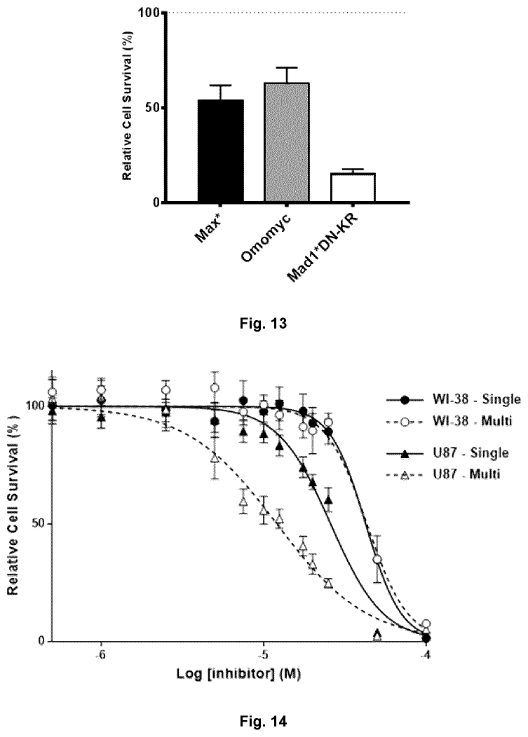

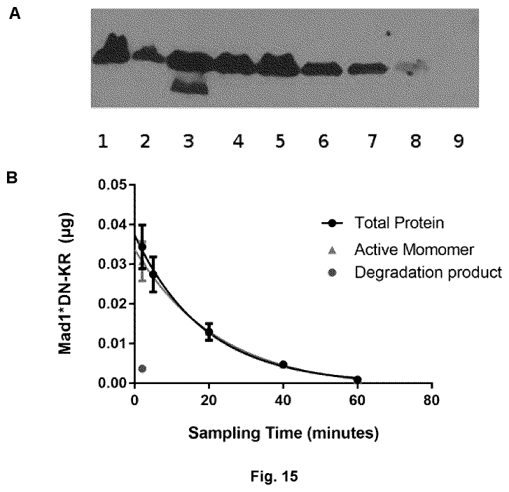

Additional comparison of Mad1*DN-KR with Max* and Omomyc in A549 cells from NSCLC confirmed the striking advantage of Mad1*DN-KR over Max* and Omomyc polypeptides. (FIG. 13). IC₅₀ experiments were further carried out on glioblastoma cells (U87) where the specificity of treatment of Mad1*DN-KR was lower (IC₅₀=25 μM) with a single treatment of 48 hours. (FIG. 14) Repeated doses allowed to increase the specificity, since it decreased the IC₅₀ to 12 μM in U87 cells. Repeated doses had no effect on the treatment of lung fibroblasts.

In order to evaluate the in vivo toxicity of the polypeptides described herein, an “up-and-down” procedure was performed to determine the acute toxicity of the compounds, in accordance with OECD recommendations in BALB/c mice. Briefly, an animal receives a dose of the compound and is observed for 48 h. If the animal does not reach the endpoint (side effects deemed important), the dose is increased by ½ log. If not, the dose is reduced by ½ log. A list of criteria for evaluating distress in animals allowed to rate the intensity of the side effects, and an experienced animal technician was responsible for the follow-up.

Once the single tolerable dose has been established, the toxicity of a repeated dose (one dose/week, 4 weeks) was evaluated in the same manner so that repeated treatment can be performed in the rest of the study.

TABLE 6
Single and multiple doses toxicity

| | Maximum tolerated dose |
|---|---|
| Single dose toxicity | 24 mg/Kg |
| Toxicity multiple doses | 24 mg/Kg |

The maximum tolerated dose (24 mg/Kg) was injected in the caudal vein of a BALB/c mouse. Blood samples of 25-50 μL (+5 μL EDTA) were taken in the saphenous vein at TO, 2, 5, 10, 20, 40 and 60 minutes to evaluate the blood residence time, as well as the final stability of our molecule.

Evaluation of blood samples 3.5 μL plasma samples
containing concentration detected in 17% EDTA

| Plasma | | | | | | |
|---|---|---|---|---|---|---|
| sampling | Total | | | Concentration | | |
| time | Products* (ug) | | | plasma (mg/mL) | | |
| (minutes) | mean | SD | N | mean | SD | N |
| 2 | 0.034404 | 0.005524 | 2 | 0.011500717 | 0.001846459 | 2 |
| 5 | 0.02743 | 0.004404 | 2 | 0.009169357 | 0.001472155 | 2 |
| 20 | 0.012943 | 0.002078 | 2 | 0.004326589 | 0.000694641 | 2 |
| 40 | 0.004716 | 0.000757 | 2 | 0.001576455 | 0.000253102 | 2 |
| 60 | 0.000896 | 0.000144 | 2 | 0.000299503 | 4.80857E−05 | 2 |
| 2 | 0.03075 | 0.004937 | 2 | 0.010279139 | 0.001650333 | 2 |
| (Active | | | | | | |
| monomer) | | | | | | |
| 2 | 0.004065 | 0.003236 | 2 | 0.001358857 | 0.001081778 | 2 |
| (Inactive | | | | | | |
| product) | | | | | | |

*in a 3.5 μL sample containing 17% EDTA

Evaluation of the blood residence half-life following the injection of a dose IV (caudal vein) of Mad1*DN-KR (24.3 mg/kg) in a healthy BALB/c mouse was performed. Blood samples were collected before injection and at various time and immediately mixed with EDTA. Plasma (supernatant) was collected by centrifugation 10 minutes at 2,000 g. 3.5 μL of plasma containing 17% EDTA of each time samples (2, 5, 10, 20, 40 and 60 minutes; wells 3 to 8 respectively) were loaded on a 15% SDS/PAGE which include a sample before injection (TO) was loaded on well 9 (FIG. 15). Quantification of Western Blot bands was done using Image J and 2 positive controls (Mad1*DN-KR) of known quantity in the well 1 (0.035 μg) and well 2 (0.0065 μg). Data were plotted and fitted using a non-linear fit (GraphPad Prism 7.0) to determine the amount (μg) in the samples that can be converted to a blood (plasma) concentration (mg/mL).

It was observed that a fraction, representing 12% of total proteins, is cleaved rapidly within the first 2 minutes of mouse blood residence. This degradation product observed at 2 minutes was analyzed by LC-MS/MS (PhenoSwitch Bioscience) and correspond to entire protein missing the 14 first N-terminal amino acids. This degradation product is undetectable after 2 minutes. The estimated half-life of total product, including degradation product is 13.00±0.037 minutes. The estimated half-life of active monomer, excluding degradation product is 15.68±0.033 minutes.

The degradation of the polypeptide was analysed by LC-MS/MS. The identified cleaved site, within the basic region is identified herein below in strikethrough:

(SEQ ID NO: 13)
60 70 80 90 100 110 [symbol]
HLRLSLKKLK RLVPLGPESS RHTTLSLLTK AKLHIKKLED
120 130 140
SNRKAIHQVD QLQREQRHLK RQLEKLGGSG C

As used herein, the term “isolated, purified or artificial polypeptide” refers to a polypeptide that has been synthesized or isolated in the laboratory, and as such, it is not “natural”. In embodiments, the polypeptides as described herein are fully artificial because they are not manufactured or expressed at all in any living organism.

It is thus described a polypeptide which comprises an amino acid sequence corresponding to a fragment of SEQ ID NO: 2 or SEQ ID NO: 4. Such polypeptides may be also referred herein as “fragment of SEQ ID NO: 2”, or “fragment of SEQ ID NO: 4”, or “Mad1 polypeptide fragment” or “fragment of Mad1”. Such fragment may be obtained by any suitable method including, but not limited to, cleaving a polypeptide comprising SEQ ID NO: 2 or SEQ ID NO: 4, laboratory amino acid synthesis of a polypeptide of a desired length, expression from a cDNA sequence encoding the desired fragment, etc.

In an embodiment, it is provided a polypeptide which comprises an amino acid sequence corresponding to a mutated version of SEQ ID NO: 2 or SEQ ID NO: 4. Such polypeptides may be also referred herein as “mutated polypeptide” or “mutated SEQ ID NO: 2” or “mutated SEQ ID NO: 4” or “Mad1 mutated polypeptide” or “mutated Mad1”. Mutated Mad1 polypeptides may be obtained by any suitable method including, but not limited to, laboratory amino acid synthesis of a polypeptide having a desired sequence, expression from a cDNA sequence encoding the desired mutated polypeptide, etc.

Therapeutically active fragments of Mad1 and/or active mutated Mad1 polypeptides include those polypeptides having at least one desirable biochemical property or effect on cancer cells, particularly on lung cancer cells, such as the biological properties listed hereinafter.

In an embodiment, the Mad1 polypeptide encompassed herein penetrate spontaneously into cells. Accordingly, the Mad1 polypeptide encompassed herein penetrates into cancer cells, escapes the endosome and accumulates to the nuclei of the cells.

A Mad1 polypeptide fragment or Mad1 mutated polypeptide may comprise at least 10, 11, 15, 25, 50, 75, 87, 100, 125, 150, 175, 200, 210, 220 or 221 contiguous amino acids of SEQ ID NO: 2. Alternatively, a Mad1 polypeptide fragment or Mad1 mutated polypeptide may comprise at least 10, 11, 15, 25, 50, 75, 87, 100, 125, 150, 175, 190, 200, 210, 211 contiguous amino acids of SEQ ID NO: 4. In an embodiment, the Mad1 polypeptide fragments or mutated Mad1 polypeptides comprise a putative Basic-Region-Helix-Loop-Helix-Leucine Zipper (b-HLH-LZ) domain and at least one desirable biochemical property or effect on cancer cells. According to one embodiment, the Mad1 polypeptide fragment or Mad mutated polypeptide comprises amino acid 58-67 of SEQ ID NO: 2.

According to a particular embodiment, the isolated, purified or artificial polypeptide is a Mad1 mutated polypeptide. In an embodiment, the Mad1 mutated polypeptide derives from SEQ ID NO: 2 or SEQ ID NO: 4 and it has been mutated to have desirable biological activity(ies), including, but not limited to, inhibition of c-Myc transcriptional activity, induction of apoptosis and/or inhibition of cancer cell proliferation.

In one particular embodiment, mutated polypeptides encompassed herein comprise a mutation at position 112 of SEQ ID NO: 2. Examples of such mutation include, but are not limited to, D112H, D112N, and D112V. In alternative embodiments, the mutated polypeptides comprise a mutation at position 102 of SEQ ID NO: 4. Examples of such mutation include, but are not limited to, D102H, D102N, and D102V.

In other embodiments, mutated polypeptides encompassed herein comprise a mutation for generating a Nuclear Localization Signal (NLS). Such NLS may be useful to maximize, facilitate or promote endosomal escape and/or to increase accumulation of the polypeptide to the nuclei of cancer cells. In one embodiment, the NLS comprises an amino acid sequence corresponding to K-(K/R)-X-(K/R). In one particular embodiment, NLS is generated by introducing mutations at positions 77 and 81 of SEQ ID NO: 2. In another embodiment, the mutation for generating the NLS is K77-K78-L79-R81. In an alternative embodiment, NLS is generated by introducing mutations at positions 67 and 71 of SEQ ID NO: 4. In a preferred embodiment, the mutation for generating the NLS is K67-K68-L69-R71.

Mutated polypeptides also encompassed herein may comprise one or more mutation useful in the laboratory (e.g. detection, tagging or labeling, ease of purification, limiting aggregation or homodimerization, etc.) In one embodiment, the mutated Mad1 polypeptide comprises a mutation at position 75 of SEQ ID NO: 2 (e.g. C75S). In one embodiment, the mutated Mad1 polypeptide comprises a mutation at position 111 SEQ ID NO: 2 (e.g. C111S). Equivalent mutations can also be done at position 65 and/or position 101 of SEQ ID NO: 4.

Mutated polypeptides further encompassed herein may also comprise one or more mutations increasing resistance to proteases degradation. In one embodiment, the mutated Mad1 polypeptide comprises a mutation at a consensus furin, PCSK7 or subtilisin-like cleavage site (RX(R/K)R). In one embodiment, the mutated Mad1 polypeptide comprises a mutation at position 69 of SEQ ID NO: 2. In one embodiment, the mutated Mad1 polypeptide comprises a substitution by a lysine (e.g. R69K). In one embodiment, the mutated Mad1 polypeptide comprises a substitution by a histidine (e.g. R69H). In another embodiment, it is encompassed the following mutated polypeptides: Mad1*DN-KR-K (SEQ ID NO: 8), Mad1*DN-KR-H (SEQ ID NO: 9), Mad1*DN-KR-TYR (SEQ ID NO: 10), Mad1*DN-R-KR (SEQ ID NO: 11), and Mad1*DN-R-KR-TYR (SEQ ID NO: 12).

In some embodiments, the mutated Mad1 polypeptide comprises an amino acid sequence as depicted in SEQ ID NOs: 5, 6, 7, 8, 9, 10, 11, 12 or 13.

According to additional embodiments, it is encompassed the use of a Mad1 polypeptide homolog, in replacement and/or in combination to a Mad1 polypeptide as defined herein. A suitable Mad1 homolog may have 70%, 75%, 80%, 85%, 90%, 95%, 97%, 99% similarity or more, over the full length of SEQ ID NO: 2 or SEQ ID NO: 4. Particularly, Mad1 homologs comprise a b-HLH-LZ domain and at least one desirable biochemical property or effect on cancer cells, such as the biological properties listed hereinafter. Suitable Mad1 fragments or mutated Mad1 could possibly be generated using such homologs. Examples of potentially useful homologs include Mad2, Mad3, Mad4 and Mnt as described hereinafter and/or variants or mutants thereof.

Mad1 fragments, mutated Mad1 polypeptides and Mad1 homologs may also be incorporated into fusion proteins comprising one or more additional functional domains (e.g. GFP, YFP, RFP, 6-HIS, GST, FLAG, HA, etc.). The polypeptides encompassed herein may also comprise a tail allowing for tagging, labeling or detection (e.g. fluorescence), purification, etc. In one embodiment, the Mad1 polypeptide comprises a 3′-terminal tail comprising the amino acid sequence GSGC.

According to present description, a Mad1 polypeptide comprises at least one, two or more, biochemical property(ies) which is(are) useful or desirable for the treatment of cancer cells. Examples of biochemical properties, but are not limited to are: spontaneously penetrate into cancer cells (e.g. lung cancer cells); localize into cytoplasmic inclusions (e.g. endosomes) and therefrom thereafter leak; localize and/or accumulate into the nucleus of cancer cells; homodimerize, heterodimerize and bind E-Box sequences in both forms; homodimerize with Max at cellular pH; sequester Max; prevent dimerization of Max with c-Myc; induce homodimerization of Mad while maintaining the ability to recognize Max; inhibit cell proliferation even more efficiently than Max*; induce apoptosis and/or halt the cell cycle; inhibit sustainably HeLa cells survival for at least 72 hours; has a selective specificity in inhibition of the proliferation inhibition of cancer cells; selectively inhibit the proliferation of lung cancer cells; recruit Histone DeACetylases (HDAC); stimulate deacetylation of histones; and revert transcriptionally active chromatin into a more condensed and inactive chromatin.

It is encompassed herein the use of a Mad1 polypeptide for inhibiting proliferation of cancer cells of a mammalian subject in need thereof, and more preferably killing cancer cells. For example, the term “mammalian subject” includes mammals in which treatment of cancer is desirable. The term “subject” includes domestic animals (e.g. cats, dogs, horses, pigs, cows, goats, sheeps), rodents (e.g. mice or rats), rabbits, squirrels, bears, primates (e.g., chimpanzees, monkeys, gorillas, and humans), transgenic species thereof, or human or precisely a human patient in need of cancer treatment. Examples of cancers, but are not limited to, are Human Lung cancer and small-cells lung cancer, Human Colon cancer, Human Glioblastoma, Human Lymphoma, Human Dermal skin fibroblast cancer, Human Myeloid fibroblast, and Breast Cancer.

Accordingly, it is provided a method for the treatment of cancer in mammalian subject in need thereof. In one embodiment, the method comprises administering to the subject a therapeutically effective amount of a Mad1 polypeptide as defined herein. In preferred embodiments, the Mad1 polypeptide is contacted with cancer cells in order to selectively inhibit proliferation of cancer cells. In an embodiment, the Mad1 polypeptide will penetrate spontaneously into the cancer cells and will cause apoptosis of the cancer cells.

Any suitable method of administration may be used including, but not limited to, rectally, intravaginally, topically, intra-nasally, intravenously, intra-arterially, intraperitoneally, intramuscularly or intratumoraly. In one embodiment, the Mad1 polypeptide is delivered directly to the lungs of the subject, for instance intranasally. In another embodiment, the Mad1 polypeptide is delivered rectally. In one embodiment, the Mad1 polypeptide is delivered intravaginally. In one embodiment, the Mad1 polypeptide is delivered topically.

Also encompassed herein is a Mad1 polypeptide as described coupled (i.e. chemically linked) to another molecule (e.g. an anticancer agent) to obtain a Mad1 conjugate allowing delivery of such molecule into desired cells.

In one embodiment, the Mad1 polypeptide comprises a free C-terminal cysteine which allow for conjugation (or derivatization) using classical thiol chemistry. This modality can be exploited by the reduction of this cysteine with reducing agents like TCEP or DTT. Completion of conjugation may be achieved with the reaction of cleavable maleimide linker such as maleimidocaproyl-valinecitrulline-p-aminobenzyl (MC-VC-PAB), where the amino acid sequence valine-citrulline can be cleaved intracellularly by a lysosomal enzyme (cathepsin), or non-cleavable maleimide linker conjugated with a desired molecule. Accordingly, it is possible to create a covalent bond between the reduced cysteine of the Mad1 polypeptide and the maleimide linker. Various molecules can be conjugated to a Mad1 polypeptide including, but not limited to, fluorophores, therapeutic compounds (e.g. small organic molecules), polypeptides or nucleic acids. In some embodiments, the Mad1 conjugate is conjugated with an anticancer agent such as auristatin E, paclitaxel, docetaxel or topotecan.

It is also provided pharmaceutical compositions comprising an effective amount a Mad1 polypeptide (and/or conjugate) as described herein. Accordingly, the pharmaceutical compositions described herein comprise at least one Mad1 polypeptide and at least one pharmaceutically acceptable carrier, diluent, vehicle or excipient.

One particular aspect concerns the use of a therapeutically effective amount of a Mad1 polypeptide (and/or conjugate) for the treatment of cancer in a mammalian subject (e.g. a human) in need thereof. Another particular aspect concerns the use of a Mad1 polypeptide and/or Mad1 conjugate for the manufacture of a medicament for the treatment of cancer(s).

The method of treatments and compositions described herein may also be used in combination with already approved anticancer therapies such as chemotherapeutic agents or cytotoxic drugs, cytokines, radiation therapy agents, etc. In one embodiment, a compound described herein (e.g. a Mad1 polypeptide) is used in combination (e.g. co-administration) or conjugated (see above) with at least one additional known chemotherapeutic agent which is currently being used or in development for treating cancer. Examples of such known compounds include, but are not limited to, decarbazine, doxorubicin, daunorubicin, cyclophosphamide, busulfex, busulfan, vinblastine, vincristine, bleomycin, etoposide, topotecan, irinotecan, taxotere, taxol, 5-fluorouracil, methotrexate, gemcitabine, cisplatin, carboplatin and chlorambucil.

### Example 1

### Selective Induction of Apoptosis in Non-Small Cell Lung Cancer (NSCLC) by Mad1 Polypeptides

The human Max b-HLH-LZ corresponding cDNA (P25912; amino acids: 22-104) and Mad1 b-HLH-LZ (Q05195; residue 57S to residue 136L) was first subcloned in the pET-3a expression plasmid (Novagen) by polymerase chain reaction (PCR) using pVZ1 p21max and pME18S-Mad1 respectively as template (both kindly provided by R. N. Eisenman, Fred Hutchinson Cancer Research Center, Seattle, Wash.) and oligonucleotides containing 5′-NdeI- and 3′-BamHI restriction sites as previously described (Montagne et al., 2012, PLoS One, 7(2): e32172). The plasmid pET3a and the PCR products were digested with NdeI and BamHI restriction enzymes before purification on agarose gel using QIAquick™ gel extraction kit (Qiagen). Ligation was achieved using T4 Ligase (NewEngland Biolabs, Pickering, ON, Canada). The absence of a stop codon allowed for a 3′-GSGC extension from pET3a vector to obtain Max*-cys and Mad1*-cys. Moreover, cysteins 75 and 111 of Mad1* were mutated for serines by site-directed PCR mutagenesis to obtain Mad1*WT-cys (C75SC111S). The D112N, D112V and D112H mutations (Mad1*DN/DH/DV) were generated by site-directed PCR mutagenesis as previously described (Montagne et al., 2008, J Mol Biol, 376: 141-152) using Mad1*WT-cys as template. The KR-variants (C75S, E77K, G81R, C111S, D112(N/H/V) were also obtained by site-directed PCR mutagenesis using 5′d(CTTGTCCCTGAAGAAGTTGAAGCGCCTGGTGCCACTG) (SEQ ID NO: 17) and 3′d(CAGTGGCACCAGGCGCTTCAACTTCTTCAGGGACAAG) (SEQ ID NO: 18) oligonucleotides and corresponding DNA template.

BL21 (DE3) pLysS competent Escherichia coli bacteria were transformed with the different Mad and Max b-HLH-LZs constructs and grown on LB agar supplemented with ampicillin (50 μg/mL) and chloramphenicol (34 μg/mL). For each mutant, one colony was selected and grown in 100 mL 2YT medium for 16 hours (pre-culture) and then diluted to 2% in a total of 5 L of 2YT medium containing ampicillin and chloramphenicol. When optical density (595 nm) reached 0.8, protein expression was induced with 0.5 mM IPTG or 0.2% L-Arabinose and bacterial cultures were grown at 37° C. overnight with agitation. Cells were harvested by centrifugation and lysed in high salt buffer. Protein extracts were centrifuged and proteins of interest were found in the inclusion bodies. The inclusion bodies were resolubilized in 6 M Urea, 0.5 M GuHCl, 100 mM NaAc buffer, pH 5.5. Proteins of interest were then purified on cation exchange chromatography columns (GE HiTrap™ SP-HP) using a salt gradient (NaCl) in citrate-phosphate buffer pH 2.8 after a wash with sodium acetate buffer pH 5.0. Highly pure fractions were desalted on HiTrap™ Desalting Columns (GE) using 0.05% H₂O.TFA and lyophilized with 15% acetonitrile. Proteins were solubilized in PBS buffer pH 7.5 and their concentration were determined using optical density at 280 nm and their respective molar extinction coefficients and compared to established references by circular dichroism spectroscopy.

To monitor for their cell penetrating properties, Mad1* polypeptides were labeled with TexasRed™ through the use of the C-terminal cysteine and maleimide chemistry. Briefly, the polypeptides were reduced in PBS buffer containing 36 mM TCEP at room temperature overnight. TexasRed™ maleimide (Sigma) was added to the proteins to a final concentration of 0.72 mg/mL and incubated for 2 hours at room temperature. Finally, the labeled-polypeptides were desalted in order to remove the free dye and lyophilized as described earlier (Montagne et al., 2012, PLoS One, 7(2): e32172).

WI-38, H23 and HeLa cells were grown in EMEM, RPMI-1640 and DMEM medium (Multicell™, Wisent) respectively, each supplemented with 10% Fetal Bovine Serum and 1× antibiotic-antimycotic (Multicell™, Wisent) at 37° C. and 5% CO₂. For immunofluorescence experiments, all three cell types were seeded on 0.5 inch microscopy cover glass slips and grown in their respective medium for 16 hours. Culture media were replaced with fresh medium containing 0-100 μM of recombinant protein and cells were allowed to grow for 1 to 2 hours.

Cells were fixed with 3% paraformaldehyde (PBS 1×) for 30 minutes, permeabilized with 0.5% Triton X-100™ for 10 minutes and blocked with 10% foetal bovine serum (FBS) for an additional 30 minutes. For detection of Max* and cleaved Caspase-3, cells were incubated with respective primary antibodies for 1 hour at room temperature. Then, cells were incubated with secondary antibodies conjugated with AlexaFluor-488™ following washing with 1% FBS. Finally, cover glass slips were mounted on microscope slides using SlowFade® with DAPI (Molecular Probes). Visualization was achieved using an inverted confocal laser-scanning microscope (FV1000™, Olympus) equipped with a PlanApo 60×/1.42 oil immersion objective (Olympus). Image acquisition and analysis was performed using version 1.6b of Olympus Fluoview™ software.

Cells were seeded in 96-well plate at 1×10³ cells per well and grown for 16 hours in their respective culture medium. Cells were then treated with our polypeptides (0-100 μM) added in fresh media and allowed to grow from 0 to 72 hours. A volume of 10 μL of Premixed WST-1 Cell Proliferation Reagent™ (Clontech) was added to each well.

Cells were incubated for 90 minutes at 37° C., 5% CO₂ and absorbance at 450 nm was measured in a multiwell plate reader.

Cells were seeded in 24-well plate at 4×10⁴ cells per well and grown for 16 hours in their respective culture medium. Detection of apoptosis was achieved using Guava Nexin Reagent™ (Millipore) according to the manufacturer's instructions. A minimum of 5000 events were acquired and analysed using Guava EasyCyte™ Mini flow cytometer and GuavaSoft™ software.

### Example 2

### Heterodimerization with Max (Max*WT) and DNA Binding are Improved by the KR Modifications

Thermal denaturations were recorded by circular dichroism (CD) at 222 nm between 5° C. and 95° C. The loss of α-Helical signal (222 nm) as temperature increases allows for the characterization of the dimeric interaction forces between the homo- and heterodimers in absence of DNA.

16p M of Mad1*DN or Mad1*DN-KR diluted in Phosphate buffer (50 mM) containing 50 mM KCl and 0.5 mM TCEP were subjected to temperature increase as the dichroic signal corresponding to α-Helical signal (222 nm) was recorded.

16 μM of Mad1*DN, Mad1*DN-KR and/or Max* diluted in Phosphate buffer (50 mM) containing 50 mM KCl and 0.5 mM TCEP were subjected to temperature increase as the dichroic signal corresponding to α-Helical signal (222 nm) was recorded. Individual denaturation curves for each protein was recorded. To compare the heterodimeric complex formation, Mad1*DN or Mad1*DN-KR were mixed with Max* at equimolar concentration (16 μM each) in the same buffer adjusted at 1 mM TCEP. The experimental denaturation curves of the complexes (Mad1*DN/Max* or Mad1DN-KR/Max*) were compared to the summation of the individual curves of each homodimer which correspond to an absence of interaction between the two proteins in solution.

A F-2500 Fluorescence Spectrophotometer (Hitachi) equipped with polarizers was used to perform fluorescence anisotropy measurements. Double stranded canonical E-box or non-specific DNA labeled with fluorescein [5′-GAG iFluorTAG CAC GTG CTA CTC-3′ (SEQ ID NO: 15) and 5′-GAG iFluorTAG GGA TCC CTA CTC-3′ (SEQ ID NO: 16) respectively] were obtained by heating the palindromic sequences, resuspended in Tris-EDTA buffer+50 mM NaCl, at 95° C. and slowly cooling to room temperature. Solutions of dsDNA (15 nM) buffered in 2 mL of 50 mM Tris, 50 mM KCl, pH 7.5 were titrated using stock solutions of protein concentrated at 15 μM and pre-equilibrated 1 h at 37° C. Each protein addition was followed by a 5-minute incubation at 37° C. in order to reach equilibrium. Anisotropy values (r) were calculated using the following equation:

$$r = \frac{{I}_{P} - {I}_{⊥}}{{I}_{P} + 2 {I}_{⊥}} \qquad ( 1 )$$

where I is the fluorescence intensity parallel or perpendicular to the incident polarization. Optimal excitation and emission wavelengths, 490 nm and 520 nm respectively, were determined using the fluorescence spectra of the labelled DNA. Slit widths were set to 10 nm.

### Example 3

### Degradation Product Identification

To identify protein bands from in-gel digestion, bands were excised in the sponsor's lab and brought to PSB. When possible, bands were cut in small cubic pieces and placed in a clear lobind 0.5 ml tube. A sufficient amount of 50 mM Tris pH 8 in 50% acetonitrile was added to the gel cubes and the samples were vortexed for 10 minutes. The supernatant was removed and thrown away. This process was repeated two more times. The gel pieces were then completely dried in a speedvac. Proteins were reduced by adding 100 μL of 10 mM DTT in 50 mM Tris pH 8 for 15 minutes at 65° C. and alkylated by adding 15 mM iodoacetamide for 1 h at room temperature in the dark. Supernatant was then removed and the gel slices were washed for 10 min vortexing in 50 mM Tris pH 8 and the supernatant was removed. The bands were dehydrated by the addition of 100 μL 50 mM Tris pH 8 in 50% acetonitrile and vortexed for 5 minutes before the supernatant was removed. This process was repeated once. 12.5 ng/μL LysC in 50 mM Tris pH 8 (freshly diluted) was added to barely cover the bands and tubes were incubated at 4° C. for 10 minutes. Additional 12.5 ng/μL LysC in 50 mM Tris pH 8 (freshly prepared) was added to completely cover the bands and tubes were incubated overnight at 37° C. with agitation. The supernatant was removed and placed in a clean tube. To the gel pieces, 30 μL of 50% ACN/5% formic acid was added and the tubes were vortexed for 20-30 min. followed by 5 min sonication. The supernatant was pooled with the digestion. This process was repeated once. Samples were then processed by reversed phase SPE and processed by LC-MS/MS.

Acquisition was performed with a ABSciex TripleTOF 5600 (ABSciex, Foster City, Calif., USA) equipped with an electrospray interface with a 25 μm iD capillary and coupled to an Eksigent pUHPLC (Eksigent, Redwood City, Calif., USA). Analyst TF 1.6 software was used to control the instrument and for data processing and acquisition. The source voltage was set to 5.2 kV and maintained at 225° C., curtain gas was set at 27 psi, gas one at 12 psi and gas two at 10 psi. Acquisition was performed in Information Dependant Acquisition (IDA) mode. Separation was performed on a reversed phase HALO C18-ES column 0.3 μm i.d., 2.7 μm particles, 150 mm long (Advance Materials Technology, Wilmington, Del.) which was maintained at 60° C. Samples were injected by loop overfilling into a 5 μL loop. For the 13 min LC gradient, the mobile phase consisted of the following solvent A (0.2% v/v formic acid and 3% DMSO v/v in water) and solvent B (0.2% v/v formic acid and 3% DMSO in EtOH) at a flow rate of 3 μL/min.

Protein identification was performed with ProteinPilot V4.5 beta (ABSciex) with the instrument preset for TripleTof5600, lodoacetamide as Cys alkylation as special factor. Thorough search with false discovery rate analysis was performed with biological modification emphasis against the mouse proteome (with custom proteins sequence added). For protein identification and data analysis global false discovery rate was set at 1% and local false discovery rate was set at 5%.

The present disclosure is not intended to be limited to the embodiments shown herein but is to be accorded the widest scope consistent with the principles and novel features disclosed herein as encompassed by the claims. It is understood that the examples and embodiments described herein are for illustrative purposes only and that various modifications or changes in light thereof will be suggested to persons skilled in the art. Accordingly, the scope of the claims should not be limited by the preferred embodiments set forth in the examples, but should be given the broadest interpretation consistent with the description as a whole.

1: An isolated polypeptide comprising the b-HLH-LZ domain of Mad1, wherein said polypeptide penetrates into cells.

2: The polypeptide of claim 1, wherein said polypeptide is (i) an amino acid sequence corresponding to a fragment of SEQ ID NO: 2 or SEQ ID NO: 4; or (ii) an amino acid sequence corresponding to a mutated SEQ ID NO: 2 or SEQ ID NO: 4.

3: The polypeptide of claim 1, comprising amino acid 58-67 of SEQ ID NO: 2.

4: The polypeptide of claim 1, comprising a mutation at position 112 of SEQ ID NO: 2.

5: The polypeptide of claim 4, wherein said mutation is selected from the group consisting of D112H, D112N, and D112V.

6: The polypeptide of claim 1, comprising a mutation at position 102 of SEQ ID NO: 4.

7: The polypeptide of claim 6, wherein said mutation is selected from the group consisting of D102H, D102N, and D102V.

8: The polypeptide of claim 1, comprising a mutation for generating a Nuclear Localization Signal (NLS).

9: The polypeptide of claim 8, wherein said NLS comprises an amino acid sequence corresponding to K-(K/R)-X-(K/R).

10: The polypeptide of claim 9, wherein said NLS is generated by introducing mutations at positions 77 and 81 of SEQ ID NO: 2.

11: The polypeptide of claim 10, wherein said NLS comprises the amino acid sequence K77-K78-L79-K80-R81, or the amino acid sequence K67-K68-L69-K70-R71.

12-13. (canceled)

14: The polypeptide of claim 1, further comprising a mutation at position 75 of SEQ ID NO: 2, a mutation at position 111 of SEQ ID NO: 2, a mutation at position 65 of SEQ ID NO: 4, a mutation at position 101 of SEQ ID NO: 4, and/or a C-terminal tail.

15: The polypeptide of claim 14, comprising a mutation C75S (SEQ ID NO: 2), a mutation C65S (SEQ ID NO: 4), a mutation C111S (SEQ ID NO: 2) and/or a mutation C101S (SEQ ID NO: 4).

16: The polypeptide of claim 14, wherein said tail comprises the amino acid sequence GSGC.

17: The polypeptide of claim 1, further comprising a mutation increasing resistance to proteases degradation.

18: The polypeptide of claim 17, comprising a mutation at a consensus furin, PCSK7 or subtilisin-like cleavage site.

19: The polypeptide of claim 17, comprising a mutation at position 69 of SEQ ID NO:2.

20: The polypeptide of claim 17, comprising a mutation R69K (SEQ ID NO:2) or a mutation R69H (SEQ ID NO: 2).

21: The polypeptide of claim 1, further comprising a mutation at position 117 of SEQ ID NO: 2, a mutation at position 119 of SEQ ID NO: 2, a mutation at position 107 of SEQ ID NO: 4, or a mutation at position 109 of SEQ ID NO: 4.

22-33. (canceled)

34: A method for treating of cancer in a subject in need thereof, said method comprising administering to said subject the polypeptide of claim 1.

35-39. (canceled)
